(19) United States
(12) Patent Application Publication
(10) Pub. No.: US 2022/0395522 A1
(43) Pub. Date: Dec. 15, 2022

(54) Methods of Use of Glucosamine for Increasing Nitrogen Retention

(71) Applicant: TSI Bioproducts (Xuzhou) Co., Ltd., Jiangsu (CN)

(72) Inventors: Ralf Jäger, Whitefish Bay, WI (US); Martin Purpura, Austin, TX (US); Jingshi Zhou, Shanghai (CN); Shixiong Ji, Shanghai (CN); Larry Kolb, Missoula, MT (US)

(21) Appl. No.: 17/832,165

(22) Filed: Jun. 3, 2022

Related U.S. Application Data
(60) Provisional application No. 63/196,425, filed on Jun. 3, 2021.

(51) Int. Cl. A61K 31/7008; A61P 1/00; A61K 45/06; A61K 9/00

(52) U.S. Cl. CPC A61K 31/7008; A61P 1/00; A61K 45/06; A61K 9/0053

(57) ABSTRACT

Methods of use and compositions comprising a source of glucosamine are disclosed. The administration of the compositions provides for favorable trends towards improvements in gastrointestinal functional outcomes. The use of the glucosamine compositions increases nitrogen retention, as well as prevent, eliminate, and/or reduce bacterial diversity within the gastrointestinal microbiome. The source of glucosamine may be non-shellfish derived, and provide significant improvements in gastrointestinal symptoms such as, stomach bloating, constipation, and hard stools.

### CROSS-REFERENCE TO RELATED APPLICATIONS

This application claims priority and is related to U.S. Provisional Application Ser. No. 63/196,425 filed on Jun. 3, 2021 and entitled Methods of Use of Glucosamine for Increasing Nitrogen Retention. The entire contents of this patent application are hereby expressly incorporated herein by reference including, without limitation, the specification, claims, and abstract, as well as any figures, tables, or drawings thereof.

### FIELD OF THE DISCLOSURE

The present disclosure relates to compositions comprising glucosamine and methods of using glucosamine to increase nitrogen retention, reduce muscle loss, and decrease bacterial diversity within the gastrointestinal microbiome of a subject.

### BACKGROUND

Glucosamine is an amino sugar found naturally in the cartilage and fluid around the joints of mammals and is also contained in crustacean exoskeletons ad well as some fungi. Glucosamine is one of the most frequently supplemented ingredients by adults in the United States, particularly due to its potential anti-inflammatory and anti-apoptotic effects on articular cartilage and bone. In clinical trials, glucosamine has frequently shown beneficial outcomes in joint pain, joint function, and other joint-related clinical outcomes. Additionally, epidemiology data has pointed to lower mortality rates in individuals who have supplemented with glucosamine. The lower mortality rate is a surprising outcome when considering glucosamine's most common application, i.e., pain relief in osteoarthritic individuals, is in a population that typically presents with multiple comorbidities.

Researchers have found under a cohort study of 77,719 people who averaged 10 years of glucosamine use, that the five-year mortality rates were significantly lower than non-glucosamine users (Pocobelli et al. Am J Clin Nutr., 2010, 91, 1791-1800, doi:10.3945/ajcn.2009.28639). In addition, a study followed 466,093 participants without cardiovascular disease (CVD) at baseline in the UK biobank for a median of seven years. Glucosamine use was associated with significantly lower risks of CVD events and death (Ma et al. Bmg 2019, 365, 11628, doi:10.1136/bmj.11628). In further examples, large prospective cohort studies involving glucosamine alone and in combination with chondroitin have suggested a reduced association of one's risk for developing colorectal cancers. Additionally, animal and human models have reported improvements in inflammatory bowel disease after glucosamine administration. In considering this data, other potential roles of glucosamine in human health and associated mechanisms of action may be possible, and other mechanisms of action, besides the proposed biochemical mechanisms related to the joints (i.e., cytokine and enzyme changes in the joints' chondrocytes, synoviocytes, and synovial fluid), should be investigated to elucidate the potential benefits seen systemically by glucosamine supplementation.

Within the human digestive tract, only 10-12% of the ingested glucosamine is absorbed. Glucosamine absorption in the digestive tract is so low that it has previously been used as a coating agent for various drugs. Due to its simplicity as an amino sugar, more than 50% of ingested glucosamine is metabolized by members of the intestinal microbiota before it is absorbed into the bloodstream. In consideration of a potential mechanism, authors have reported that glucosamine (and chondroitin) may function in various roles in the production of anti-inflammatory compounds that are known to be increased in various inflammatory and metabolic diseases.

However, evidence examining the potential ability of glucosamine to change the abundances of various strains and species of bacteria in the gastrointestinal microbiome is limited. A recent study observed that oral co-administration of glucosamine hydrochloride (derived from crab and shrimp) and chondroitin sulfate significantly modulated nine different genera after a 14-day supplementation using a placebo-controlled, crossover design (Navarro et al. Microorganisms 2019, 7, doi:10.3390/microorganisms7120610). Within the study, abundances of Lachnospiraceae, Prevotellaceae, and Desulfovibrio were increased while Bifidobacterium and Christensenellaceae were decreased. However, there is a lack of clinical studies investigating the ability of glucosamine to independently impact changes in stool, gastrointestinal symptoms and changes in the quantity and function of bacteria in the microbiome in comparison to a control group. Accordingly, there remains a need for additional compositions and methods comprising glucosamine for supplementation on changes in the fecal microbiome, stool function, and gastrointestinal symptoms.

Other objects, advantages and features of the present disclosure will become apparent from the following specification taken in conjunction with the accompanying figures.

### SUMMARY

In Example 1, a method of increasing nitrogen retention in a subject comprises administering to the subject a therapeutically effective amount of a composition comprising a glucosamine, wherein the administering of the composition increases nitrogen retention in the subject.

Example 2 relates to the method according to Example 1, wherein the subject is administered a total daily dose of between about 500 mg and about 5,000 mg of the glucosamine.

Example 3 relates to the method according to Example 2, wherein the composition is administered one or more times per day.

Example 4 relates to the method according to Example 2, wherein the composition is administered up to three times per day.

Example 5 relates to the method according to Example 2, wherein the composition is administered to the subject for a period of at least 21 days.

Example 6 relates to the method according to Example 1, wherein the administering of the composition reduces amino acid excretion via stools of the subject.

Example 7 relates to the method according to Example 1, wherein the administering of the composition reduces a rating of stomach bloating by the subject according to Gastrointestinal Symptom Rating Scale 7 (GSRS).

Example 8 relates to the method according to Example 1, wherein the glucosamine is not derived from shellfish.

Example 9 relates to the method according to Example 1, wherein the composition is not co-administered with chondroitin or green lipped muscle extract to the subject.

In Example 10, a method of preventing, eliminating, and/or reducing bacterial diversity within a gastrointestinal microbiome of a subject comprises administering to the subject a therapeutically effective amount of a composition comprising a glucosamine, wherein the administering of the composition prevents, eliminates, and/or reduces bacterial diversity within the gastrointestinal microbiome of the subject.

Example 11 relates to the method according to Example 10, wherein the subject is administered a total daily dose of between about 500 mg and about 5,000 mg of the glucosamine.

Example 12 relates to the method according to Example 11, wherein the composition is administered one or more times per day.

Example 13 relates to the method according to Example 11, wherein the composition is administered up to three times per day.

Example 14 relates to the method according to Example 11, wherein the composition is administered to the subject for a period of at least 21 days.

Example 15 relates to the method according to Example 10, wherein the administering of the composition reduces the percent abundance of Pseudomonadaceae, Peptococcaceae, Bacillaceae, or a combination thereof within the gastrointestinal microbiome of the subject.

Example 16 relates to the method according to Example 10, wherein the administering of the composition reduces a rating of stomach bloating by the subject according to Gastrointestinal Symptom Rating Scale 7 (GSRS).

Example 17 relates to the method according to Example 10, wherein the glucosamine is not derived from shellfish.

Example 18 relates to the method according to Example 10, wherein the composition is not co-administered with chondroitin or green lipped muscle extract to the subject.

In Example 19, an oral composition for improving gastrointestinal function in a subject comprises a source of glucosamine selected from the group consisting of glucosamine hydrochloride, glucosamine sulfate, and N-acetylglucosamine; and a pharmaceutically acceptable carrier, wherein the source of glucosamine is not derived from shellfish.

Example 20 relates to the composition according to Example 19, wherein the composition is substantially free of chondroitin and green lipped muscle extract.

While multiple embodiments are disclosed, still other embodiments of the present disclosure will become apparent to those skilled in the art from the following detailed description, which shows and describes illustrative embodiments of the disclosure. Accordingly, the figures and detailed description are to be regarded as illustrative in nature and not restrictive.

### BRIEF DESCRIPTION OF THE FIGURES

FIG. 1 shows a flowchart of the experimental design that was followed by the participants. The flowchart identifies each of the tasks required for each of the four office visits, as well as the timing of glucosamine administration.

FIG. 2 shows a flowchart of the Consolidated Standards of Reporting Trials (CONSORT) Diagram procedures.

FIG. 3A shows a graph with the Faith's phylogenetic diversity (PD) measurements of the bacterial contents in fecal samples of the participants, both before and after placebo (PLA) and glucosamine (GLU) consumption.

FIG. 3B shows a graph with the Principal Components Analysis (PCoA) of bacterial diversity according to an unweighted UniFrac metric. Fecal samples from each individual participant are connected via black lines within the graph.

FIG. 4 shows a stacked bar chart with the relative percent abundance of bacteria for both pre and post administration of glucosamine (GLU) or placebo (PLA). Seventeen of the most abundant bacterial families are noted, with all other taxa grouped under the annotation of “Other.”

FIG. 5 shows a graph of the percent abundance of bacteria taxa present in fecal contents of participants both before and after administration of glucosamine (GLU) and placebo (PLA). FIG. 5 shows the results for Enterococcaceae, Lactococcus, Pseudomonadaceae, Peptococcaceae, and Bacillaceae.

Various embodiments of the present disclosure will be described in detail with reference to the figures. Reference to various embodiments does not limit the scope of the disclosure. Figures represented herein are not limitations to the various embodiments according to the disclosure and are presented for exemplary illustration of the disclosure.

### DETAILED DESCRIPTION

The present disclosure relates to the impact of glucosamine supplementation on changes in the fecal microbiome, stool function, and gastrointestinal symptoms. In some aspects, the glucosamine is a non-shellfish-derived glucosamine. The composition and methods of the present disclosure yield significant improvements in gastrointestinal function and can be responsible for significant changes in genera of bacteria found in the fecal microbiome of a subject.

### Definitions

So that the present disclosure may be more readily understood, certain terms are first defined. Unless defined otherwise, all technical and scientific terms used herein have the same meaning as commonly understood by one of ordinary skill in the art to which embodiments of the disclosure pertain. Many methods and materials similar, modified, or equivalent to those described herein can be used in the practice of the embodiments of the present disclosure without undue experimentation, the preferred materials and methods are described herein. In describing and claiming the embodiments of the present disclosure, the following terminology will be used in accordance with the definitions set out below.

The term “about,” as used herein, refers to variation in the numerical quantity that can occur, for example, through typical measuring techniques and equipment, with respect to any quantifiable variable, including, but not limited to, mass, volume, time, distance, wave length, frequency, voltage, current, and electromagnetic field. Further, given the solid and liquid handling procedures used in the real world, there is certain inadvertent error and variation that is likely through differences in the manufacture, source, or purity of the ingredients used to make the compositions or carry out the methods and the like. The term “about” also encompasses these variations. Whether or not modified by the term “about,” the claims include equivalents to the quantities.

The term “actives” or “percent actives” or “percent by weight actives” or “actives concentration” are used interchangeably herein and refers to the concentration of those ingredients involved expressed as a percentage minus inert ingredients such as water or salts. It is also sometimes indicated by a percentage in parentheses, for example, “chemical (10%).”

As used herein, the term “glucosamine” is understood to refer to glucosamine, derivatives of glucosamine, analogs of glucosamine, and metabolites of glucosamine, unless otherwise indicated.

As used herein, the term “substantially free” refers to compositions completely lacking the component or having such a small amount of the component that the component does not affect the performance of the composition. The component may be present as an impurity or as a contaminant and shall be less than 0.5 wt-%. In another embodiment, the amount of the component is less than 0.1 wt-%, and in yet another embodiment, the amount of component is less than 0.01 wt-%.

The term “weight percent,” “wt-%,” “percent by weight,” “% by weight,” and variations thereof, as used herein, refer to the concentration of a substance as the weight of that substance divided by the total weight of the composition and multiplied by 100. It is understood that, as used herein, “percent,” “%,” and the like are intended to be synonymous with “weight percent,” “wt-%,” etc.

The embodiments of this disclosure are not limited to particular methods and compositions which can vary and are understood by skilled artisans. It is further to be understood that all terminology used herein is for the purpose of describing particular embodiments only and is not intended to be limiting in any manner or scope. For example, as used in this specification and the appended claims, the singular forms “a,” “an” and “the” can include plural referents unless the content clearly indicates otherwise. Further, all units, prefixes, and symbols may be denoted in its SI accepted form.

Numeric ranges recited within the specification are inclusive of the numbers defining the range and include each integer within the defined range. Throughout this disclosure, various aspects of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 1½, and 4¾. This applies regardless of the breadth of the range.

The methods and compositions of the present disclosure may comprise, consist essentially of, or consist of the components and ingredients of the present disclosure as well as other ingredients described herein. As used herein, “consisting essentially of” means that the methods, systems, apparatuses and compositions may include additional steps, components, or ingredients, but only if the additional steps, components, or ingredients do not materially alter the basic and novel characteristics of the claimed methods and compositions.

Methods and Compositions

The present disclosure provides for methods of use related to compositions comprising glucosamine as described herein. As provided herein, the methods of use provide for significant improvements in markers of gastrointestinal symptoms and provide for improved functional outcomes. In certain aspects, the compositions and methods disclosed herein provide for significant reduction in stomach bloating. In aspects, the compositions reduce the rating of stomach bloating according to the Gastrointestinal Symptom Rating Scale 7 (GSRS7). In further aspects, the administration of the disclosed compositions provides for beneficial changes in constipation and hard stools, which favor the use of the glucosamine compositions as supplementation. The compositions and methods may provide these beneficial outcomes without any changes on stool quality. In further aspects, modest but significant alterations in bacterial composition in the stools of a subject may be observed after glucosamine administration, including, but not limited to, reductions in alpha diversity and reduced proportions of several bacterial taxa. These beneficial changes may occur in the absence of observable modifications to the fecal metabolome of the subject.

While an understanding of the mechanism is not necessary to practice the present disclosure and while the present disclosure is not limited to any particular mechanism of action, it is contemplated that, in some embodiments, the benefits of the compositions and uses thereof comprising glucosamine may be partially related to the modification of the intestinal environment of a subject, including, but not limited to, the gut microbiota. In certain embodiments, the subject is a healthy individual, however, the methods and compositions disclosed herein are not limited to administration to only healthy subjects.

In certain embodiments, the present disclosure provides for methods of increasing nitrogen retention in a subject. In further aspects, the method reduces amino acid excretion via stools of the subject. In some embodiments, the administration of the compositions provides for reduced individual, total branched-chain, and total amino acid excretion, with no detection of glucosamine within the stool. As described herein, a reduction of total amino acids (as well as additional components such as branched-chain amino acids (BCAA) and/or glutamate) within the stools of a subject may be an indicator of increased absorption of those nutrients, increased utilization by microbes, or decreased production, assuming that some amino acids are provided directly from gut bacteria of the subject. In further aspects, there is an established link between reduced nitrogen excretion, such as through fecal material, and reduced muscle protein breakdown. While an understanding of the mechanism is not necessary to practice the present disclosure, and while the present disclosure is not limited to any particular mechanism of action, it is contemplated that, in some embodiments, as increased muscle breakdown results in the excretion of amino acids in the feces, less amino acids (or nitrogen) in the stool signals that more amino acids are retained in the body. In certain embodiments, the methods and compositions disclosed herein increase nitrogen retention, and thus, reduced muscle protein breakdown within a subject.

In further aspects, the methods and compositions further provide for loss of fat in a subject. The loss of fat may occur concurrently with the maintenance of muscle mass as disclosed herein. The methods and compositions may further provide for appetite control, weight management, or a combination thereof. In certain aspects, the administration of the composition does not have effects on fecal short-chain fatty acid levels of the subject.

The present disclosure further provides for methods of preventing, eliminating, and/or reducing bacterial diversity within a gastrointestinal microbiome of a subject. In certain embodiments, the disclosure provides for the reduction in bacterial alpha-diversity (a metric of the total bacterial contents in a fecal material) within a subject. In some aspects, the methods and compositions do not have an effect on bacterial beta-diversity. In some embodiments, the methods and compositions provide for significant reductions in the proportions of bacteria, including, but not limited to, Pseudomonadaceae, Peptococcaceae, and Bacillaceae, in fecal matter of a subject. In further embodiments, levels of Bifidobacterium and Christensenellaceae may also be reduced with the administration of the compositions disclosed herein.

The compositions described herein may be administered to a subject using any amount and any route of administration effective for improving the functional outcomes of the gastrointestinal biome of a subject. The exact amount required will vary from subject to subject, depending on the species, age, and general condition of the subject, the severity of the gastrointestinal symptoms, the particular formulation, its mode of administration, its mode of activity, and the like.

In some embodiments, the composition may be administered according to one or more administration routes. In some embodiments, administration is oral, enteral, transdermal, sublingual, nasogastric, injectable, intravenous, intramuscular, subcutaneous, intradermal, rectal, buccal, intra-abdominal, intragastric, intranasal, or intraduodenal. Dosage forms include tablets, pills, troches, dispersions, suspensions, solutions, capsules, creams, ointments, aerosols, beverages, juices, pastries and other traditional food forms, and the like. In certain embodiments, it may be advantageous that the compositions described herein be administered orally.

In some embodiments, the compositions in accordance with the present disclosure may be administered at dosage levels sufficient to deliver a therapeutically effective amount of active ingredient to the subject. In an aspect, the compositions include from about 500 mg to about 5000 mg, from about 750 mg to about 5000 mg, from about 1000 mg to about 5000 mg, from about 1000 mg to about 4000 mg, from about 2000 mg to about 4000 mg, or about 3000 mg of glucosamine. In addition, without being limited according to the invention, all ranges recited are inclusive of the numbers defining the range and include each integer within the defined range.

In some embodiments, the compositions are provided in one or more doses and are administered one or more times to the subject. In some embodiments, the compositions are provided in only a single administration per day. In other embodiments, the compositions are provided according to a dosing schedule that include two or more administrations per day. In some embodiments, the composition is administered up to three times per day. Each administration may be at the same dose or may be different from a previous and/or subsequent dose. In addition, without being limited according to the invention, all ranges recited are inclusive of the numbers defining the range and include each integer within the defined range.

In some embodiments, the compositions may be delivered for a duration of about 3 days to about 365 days, about 5 days to about 365 days, about 10 days to about 365 days, about 14 days to about 365 days, about 21 days to about 365 days, about 3 days to about 100 days, about 5 days to about 60 days, about 7 days to 30 days, about 14 days to about 30 days, or about 21 days to 28 days. In some embodiments, the composition may be delivered for a duration of at least 21 days. In addition, without being limited according to the invention, all ranges recited are inclusive of the numbers defining the range and include each integer within the defined range.

The compositions in accordance with the present disclosure comprises glucosamine. In some embodiments, the glucosamine includes derivatives of glucosamine, analogs of glucosamine, and metabolites of glucosamine. In certain embodiments, the glucosamine comprises glucosamine hydrochloride, glucosamine sulfate (including, but not limited to, glucosamine sulfate, glucosamine sulfate sodium chloride, glucosamine sulfate potassium chloride), N-acetylglucosamine, or a combination thereof. In some aspects, the glucosamine is glucosamine hydrochloride. In some embodiments, the glucosamine is not limited to any particular source. The glucosamine may be derived from shellfish or via fermentation (i.e., not derived from shellfish). In other embodiments, the glucosamine is not derived from shellfish. In aspects, glucosamine that is not derived from shellfish provides benefits of being safely taken by subjects having allergies, including, but not limited to, seafood allergies, as well as subjects having specific dietary restrictions.

In certain embodiments, the compositions may optionally be co-administered with an additional therapeutic agent. In some embodiments, the additional therapeutic agent comprises chondroitin (including, but not limited to, chondroitin sulfate or chondroitin sulfate sodium), green lipped muscle extract, β-hydroxy-β-methylbutyrate (HMB), type II collagen, or a combination thereof. In other embodiments, the composition is not co-administered with chondroitin. In other embodiments, the composition is not co-administered with green lipped muscle extract. In further aspects, the compositions of the disclosure may be substantially free of, or completely free of, chondroitin and/or green lipped muscle extract.

In certain embodiments, the compositions may further comprise a pharmaceutically acceptable excipient or carrier, which, as used herein, includes any and all solvents, dispersion media, diluents, or other liquid vehicles, dispersion or suspension aids, surface active agents, isotonic agents, thickening or emulsifying agents, preservatives, solid binders, lubricants, flavors, colors, sweeteners, and the like, as suited to the particular dosage form desired. In embodiments, pharmaceutically acceptable excipients or carriers used in the manufacture of the disclosed compositions include, but are not limited to, inert diluents, dispersing and/or granulating agents, surface active agents and/or emulsifiers, disintegrating agents, binding agents, preservatives, buffering agents, lubricating agents, flavoring agents, dyes, coloring agents, and/or oils. Such excipients and carriers may optionally be included in the compositions.

All publications and patent applications in this specification are indicative of the level of ordinary skill in the art to which this disclosure pertains. All publications and patent applications are herein incorporated by reference to the same extent as if each individual publication or patent application was specifically and individually indicated as incorporated by reference.

### EXAMPLES

Embodiments of the present disclosure are further defined in the following non-limiting Examples. It should be understood that these Examples, while indicating certain embodiments of the disclosure, are given by way of illustration only. From the above discussion and these Examples, one skilled in the art can ascertain the essential characteristics of the present disclosure, and without departing from the spirit and scope thereof, can make various changes and modifications of the embodiments to adapt it to various usages and conditions. Thus, various modifications of the embodiments of the disclosure, in addition to those shown and described herein, will be apparent to those skilled in the art from the foregoing description. Such modifications are also intended to fall within the scope of the appended claims.

### Example 1

### Experimental Design

A randomized, double-blind, placebo-controlled, cross-over study design was conducted on healthy males and females. Participants were instructed to report to a laboratory after observing an overnight (8 to 10-hour) fast while maintaining adequate hydration. Participants were asked to abstain from exercise and avoid alcohol and caffeine consumption for 12 hours prior to each study visit. All participants first completed an initial eligibility visit during which a comprehensive medical history, consent document, and body mass index (BMI) assessment were completed. Results from the BMI assessment was used to finalize eligibility. Once deemed eligible, participants were provided with supplies to begin the study protocol.

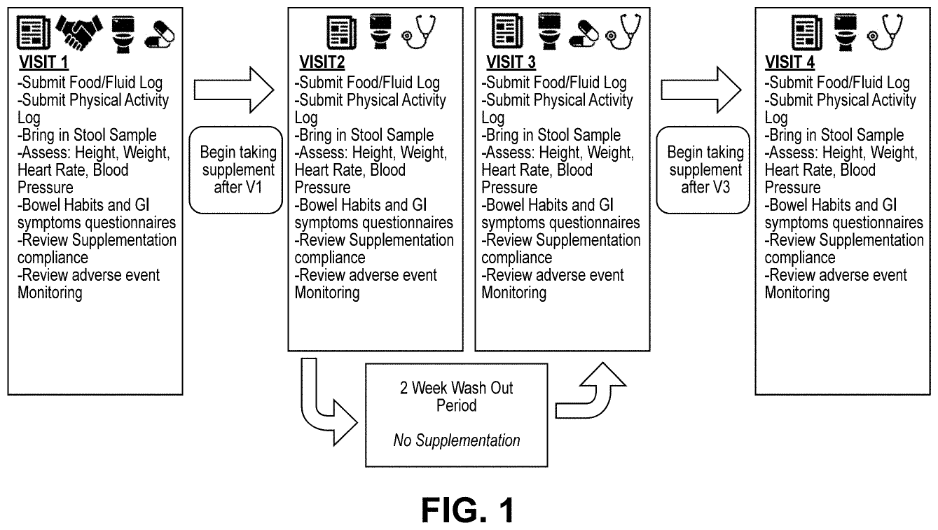

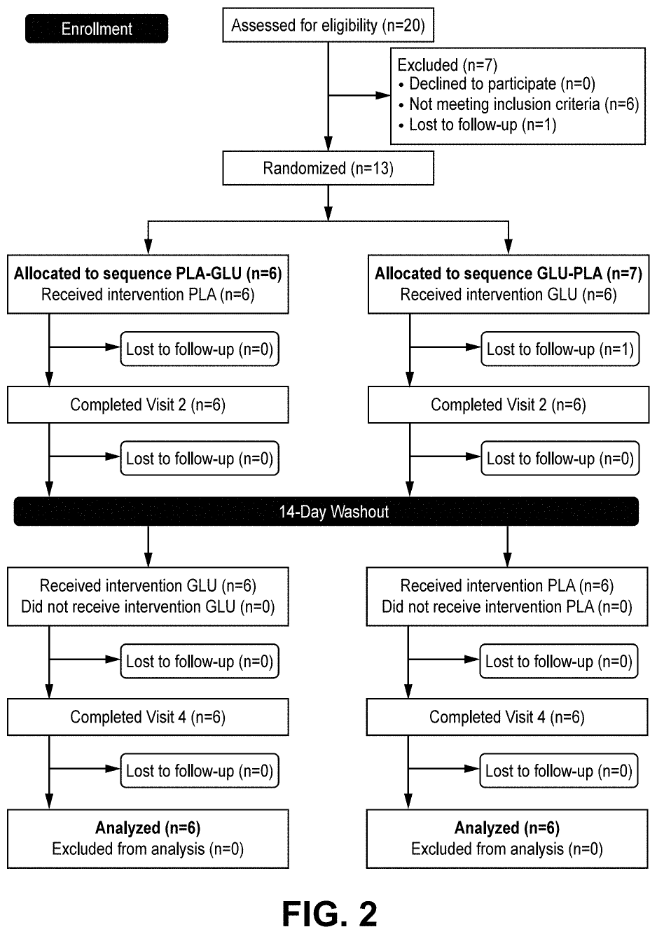

The remainder of the study consisted of two identical patterns of testing with the exception of what supplementation protocol was followed. FIG. 1 provides a flowchart of the experimental design. Prior to each visit, food and physical activity logs were completed encompassing four days prior to the scheduled laboratory visit. The four days consisted of two weekend days and two weekdays. Each participant reported to the laboratory for a baseline measurement of height, weight, heart rate, and blood pressure. During this visit, participants completed the Bristol Stool Chart and Gastrointestinal Symptom Rating Scale (GSRS) to assess bowel habits and potential gastrointestinal symptoms. Participants were given instructions on how to collect a stool sample and were instructed to do so within 24 hours of each planned study visit. At the completion of this study visit, participants were provided with their first assigned supplement (either PLA [placebo] or GLU [glucosamine]) and instructed to begin supplementation 21 days prior to their second visit. Participants were instructed to collect their second stool sample on the day of their final supplementation dose. On the morning of their second study visit, participants reported to the laboratory after following identical pre-study instructions as previous visits (i.e., fasting, abstention from caffeine and exercise). Study participants again completed assessments for height, weight, heart rate, blood pressure, and questionnaires regarding their bowel habits and gastrointestinal symptoms while also providing their second stool sample and turning in completed food and activity logs. A washout period lasting a minimum of 14 days and a maximum of 21 days separated each phase of the crossover as shown in FIG. 1. As shown in FIG. 2, a Consolidated Standards of Reporting Trials (CONSORT) diagram for all study recruitment randomization and project completion is provided.

Data collection commenced in January 2019 and was completed in June 2019. A total of six male (32.0±9.6 years, 180.5±10.0 cm, 81.0±11.3 kg, 24.8±2.8 kg/m²) and five female (35.0±5.4 years, 166.5±10.1 cm, 71.1±13.8 kg, 25.6±3.8 kg/m²) participants completed the study protocol. Prior to participation, participants provided their signed informed consent using an IRB approved consent document (Protocol #IRB-19-8, Approval Date Oct. 18, 2018). Participants were randomized into the study protocol if they were between the ages of 18-50 years, had a body mass index between 18.5-27 kg/m², weight stable for the past three months (less than 5% variation in body mass), and free from all cardiovascular, pulmonary, autoimmune, musculoskeletal, gastrointestinal, psychological, or other diseases or disorders, as reported in their medical history. Participants could not have been currently taking any antibiotics, probiotics, or indicated they were actively trying to lose weight which included participating in diets that would impact study outcomes (ketogenic or low carbohydrate diet). Alternatively, participants were excluded if they were: diagnosed with or were being treated for celiac disease, lactose intolerance, digestive insufficiencies, or other gastrointestinal complications such as irritable bowel syndrome, ulcerative colitis, etc.; a current smoker or had quit within the past six months; currently using anabolic steroids or any illicit or recreational drugs; or currently taking any antibiotic or medication known to impact study outcomes. Participants who reported consuming a probiotic or other form of any dietary supplement purported to impact digestion or gut health were required to stop taking that supplement for 30 days before beginning the study trial.

In a double-blind manner, a glucosamine supplement (GLU) or a maltodextrin placebo (PLA) was distributed in identical opaque capsules to all participants. On a daily basis, participants were instructed to ingest three capsules, each containing 1,000 mg of glucosamine hydrochloride (GlucosaGreen®, TSI Group Ltd. MT, USA) or a placebo (maltodextrin) identical in appearance for a total of 21 days. Each visit at the beginning of the supplementation regimen (Visits 1 and 4, respectively; See FIG. 1) occurred on or before the first day of supplementation while the final day of supplementation occurred the day before each participant's follow-up visit for that study period (Visits 2 and 4, respectively). Participants were instructed to ingest the supplement 15-30 minutes before breakfast with eight fluid ounces of cold water. In the case of a missed dose, participants were instructed to consume the missed dose as soon as possible with a meal. A maltodextrin placebo was used due to previous research that demonstrated a lack of changes in fecal microbiome content after 7.5 and 15 grams of resistant maltodextrin was ingested. The dose of maltodextrin used in the following examples was two to five-fold lower, respectively, than these investigations.

Primary outcomes were considered to be between group differences between the components of the GSRS and the Bristol Stool Chart, with secondary outcomes being the metabolomic analysis. No between-gender analysis was completed due to low sample size. All analysis was completed with both genders collapsed using Microsoft Excel and the Statistical Package for the Social Sciences (v23; SPSS Inc., Chicago Ill.). Before any statistical tests were completed, normality was assessed for all dependent variables. All non-normal data was log-transformed and then analyzed using both parametric and non-parametric approaches. For all dependent measures, descriptive statistics are presented herein as mean±standard deviations. When the normality assumption was met, paired samples t-tests were computed using the post—pre changes for all variables and data was presented as means±standard deviation. When the normality assumption was not met, Wilcoxon signed rank tests were completed using post—pre changes for all variables. For all statistical tests provided within the following examples, data was considered statistically significant when the probability of type I error was 0.05 or less. A p-value between 0.05-0.10 was considered to be a statistical trend. Using this approach, no situations arose where the final statistical decision was different whether a parametric or non-parametric approach was used. As such, between-group effect sizes, p-values, and 95% confidence intervals were computed to accompany traditional hypothesis testing outcomes.

To determine significant differences between bacterial alpha diversity metrics (the measure of microbiome diversity within a single sample), a paired t-test was used between alpha diversity values of different sample groups (p<0.05). Significant differences in beta diversity (the measure of similarity or dissimilarity of multiple samples) between groups was determined using the qiime diversity beta-group-significance command in QIIME2 as well, which performs pairwise Permutational Multivariate Analysis of Variance (PERMANOVA) tests on beta diversity distance matrices (p<0.05). Differential abundance of taxa between sample groups were evaluated using Multivariate Analysis by Linear Models (MaAsLin) in R version 4.0.2 using the Maaslin2 command in the Maaslin2 R library on rarified taxonomy data generated by QIIME2 (p<0.05). Additional evaluation of differential abundance of taxa was performed using Analysis of Composition of Microbiomes (ANCOM) using the qiime composition ancom command after collapsing the feature table to the family level (level 5) using the qiime taxa collapse command on QIIME2. Data visualization was performed using the ggplot2 package, with data frame manipulation commands from the dplyr package, within the tidyverse collection of R packages.

### Example 2

### Demographics, Hemodynamics, and Dietary Intake

Height and weight were measured for every participant without shoes and in minimal clothing using a standardized wall-mounted stadiometer (Tanita, model #HR-200, Tokyo, Japan) and a self-calibrating digital scale (Tanita, model #BWB-627A Class III, Tokyo, Japan). Prior to heart rate and blood pressure measurements, participants rested in a seated position for five minutes. Heart rate was measured on the right arm at the radial artery by placing the index and middle fingers two centimeters proximal to the styloid process of the radius by a trained research assistant. The number of heart beats was counted over a 30-second period and multiplied by two. Blood pressure was measured on the right arm using a standard blood pressure cuff and sphygmomanometer at the brachial artery. Two measurements were taken and averaged. The right arm was positioned at the level of the heart with the distal edge of the cuff placed two centimeters above the bend in the elbow prior to the cuff being inflated to a value that was 30 millimeters of mercury (mmHg) higher than the participant's self-reported blood pressure. The cuff was deflated at approximately 3 mmHg per second until the first and fourth Korotkoff sounds were detected. Both values associated with these events were recorded and used for the blood pressure assessment.

Further, study participants completed assessments of food intake and physical activity at the beginning and end of each phase of testing. Intake was reported over a four-day period prior to in-lab assessments, encompassing two weekdays and two weekend days. All activities of daily living as well as exercise and sleep were self-reported by participants on a 24-hour fitness log. All food intake encompassing food type, preparation style, and amount were self-reported on paper by participants for the four-day assessment period.

Compliance for the collection of dietary and physical activity data were recorded throughout the protocol. Overall compliance across both conditions was calculated to be 86% with compliance exhibited by individual participants ranging from 50-100%. One male and one female participant during the glucosamine condition failed to turn in any diet or physical compliance information. One female during the glucosamine condition, did not turn in food or physical activity records.

Table 1 contains the results of the body mass, sleep, physical activity, hemodynamic, and dietary data for all participants. No between-group or within-group differences were identified for any of these variables.

TABLE 1
Participant Demographics, Hemodynamics, and Dietary Intake

| | | | | | | | PLA vs. |
|---|---|---|---|---|---|---|---|
| | PLA | | | GLU | | | GLU |
| | Pre | Post | p | Pre | Post | p | p |
| Body Mass | 77.0 ± 13.3 | 76.6 ± 12.6 | 0.15 | 76.5 ± 12.7 | 76.8 ± 12.5 | 0.60 | 0.43 |
| (kg) | | | | | | | |
| Heart Rate | 66.8 ± 10.4 | 65.8 ± 7.9 | 0.69 | 66.8 ± 7.7 | 63.1 ± 8.1 | 0.22 | 0.62 |
| (beats/min) | | | | | | | |
| Systolic BP | 123.6 ± 12.2 | 116.7 ± 13.3 | 0.04 | 121.7 ± 14.2 | 119.4 ± 10.8 | 0.60 | 0.09 |
| (mmHg) | | | | | | | |
| Diastolic BP | 74.3 ± 7.0 | 72.1 ± 8.2 | 0.02 | 73.9 ± 9.3 | 77.0 ± 9.8 | 0.33 | 0.11 |
| (mmHg) | | | | | | | |
| Physical | 51 ± 29 | 53 ± 29 | 0.90 | 45 ± 24 | 32 ± 20 | 0.33 | 0.56 |
| Activity | | | | | | | |
| (min/day) | | | | | | | |
| Energy | 2037 ± 621 | 2091 ± 742 | 0.32 | 2146 ± 663 | 2085 ± 530 | 0.82 | 0.92 |
| (kcal/day) | | | | | | | |
| Carbohydrate | 213 ± 50 | 210 ± 91 | 0.76 | 209 ± 69 | 189 ± 36 | 0.80 | 0.82 |
| (g/day) | | | | | | | |
| Protein | 124 ± 68 | 118 ± 51 | 0.78 | 124 ± 52 | 123 ± 46 | 0.93 | 0.90 |
| (g/day) | | | | | | | |
| Fat (g/day) | 75 ± 28 | 87 ± 35 | 0.08 | 89 ± 14 | 91 ± 36 | 0.35 | 0.86 |

### Example 3

### Bristol Stool Chart and Gastrointestinal Symptom Rating Scale

Participants completed electronic (Qualtrics, Inc.) versions of the Bristol Stool Chart and the Gastrointestinal Symptoms Rating Scale (GSRS) at four time points throughout the entire study protocol. The first two questionnaires were completed before and after the first supplementation period while the final two questionnaires were collected before and after the second supplementation period.

The Bristol Stool Chart requires participants to classify their bowel movements into one of seven different types of stool. Type 1 was classified as being “Separate hard lumps, like nuts (hard to pass)”, Type 2 being “Sausage-shaped, lumpy, uncomfortable to pass”, Type 3 being “Like a sausage, with cracks on its surface”, Type 4 being “Like a sausage or snake, smooth and soft”, Type 5 being “Soft blobs with clear-cut edges, passes easily”, Type 6 being “Fluffy pieces with ragged edges, a mushy stool”, and Type 7 being “Watery, no solid pieces, entirely liquid”. The Gastrointestinal Symptoms Rating Scale (GSRS), a 15-item clinical rating scale was completed a total of four times to assess symptoms related to gastrointestinal distress during the past week. Each item was scored on a seven-point scale where 1=No Discomfort at all; 2=Minor Discomfort; 3=Mild Discomfort; 4=Moderate Discomfort; 5=Moderately Severe Discomfort; 6=Severe Discomfort; and 7=Very Severe Discomfort. The GSRS encompasses areas such as: pain and discomfort in upper abdomen, heartburn, acid reflux, hunger pangs, nausea, stomach rumbling, bloating, burping, passing gas or flatus, constipation, diarrhea, hard stools, urgent need to have a bowel movement, and sensation of not completely emptying the bowels.

Stool specimens were collected at four time points throughout the entire study protocol. The first two samples were collected before and after the first supplementation period while the final two samples were collected before and after the second supplementation period. All stool specimens were collected no more than 24-hours prior to that study participant being scheduled to complete their laboratory visit. All samples were immediately frozen after sample collection by the study participants. Participants were instructed to keep their samples in the freezer and to remove them at the latest possible time to minimize thawing during transfer to the laboratory for storage. Participants were provided with all materials necessary to collect, store, and transport the stool specimen including commode fecal collection system, fecal collection tubes with screw top lids and scoops attached, disposable gloves, freezer packs, specimen labels, and transfer containers. Participants were instructed to raise the toilet seat and place the stool collection frame on the back of the toilet bowl and then lower the toilet seat down and place collection bowl in frame. Participants then sat on the toilet and collected a stool sample into the container. Participants were instructed, if possible, to not urinate into the collection container. Participants then extracted a representative stool sample and filled each of the two provided sample collection tubes. Tubes were then secured, labeled, and frozen. All remaining stool was discarded into the toilet and flushed. Upon arrival in the laboratory each sample was immediately stored at −80° C.

Compliance for the collection of fecal samples was recorded throughout the protocol. Overall compliance was calculated to be 86% with compliance exhibited by individual participants ranging from 50-100%. One male and one female participant during the glucosamine condition failed to turn in any fecal sample compliance information.

With regard to adverse events, no formalized approach was completed primarily because gastrointestinal symptoms were being recorded at four times throughout the study protocol. Nonetheless, participants were asked during each visit if they experienced any other adverse events that they needed to report. No other adverse events were reported.

As shown in Tables 2A and 2B, no significant changes in Bristol Stool chart ratings were observed within the PLA (p=0.10) and GLU (p=0.10) or between group (p=0.20).

As further shown in Tables 2A and 2B, significantly greater increases in stomach bloating (GSRS7) were reported in the PLA group when compared to changes observed in GLU (condition×time, p=0.03). Additionally, stomach bloating ratings in PLA significantly increased (p=0.03) from baseline while no changes were observed (p=0.32) in GLU. Both ‘Constipation’ (GSRS10, p=0.10) and ‘Hard Stools’ (GSRS13, p=0.08) exhibited a trend for the changes observed during supplementation to be different between groups. Within-group changes for ‘Constipation’ indicated a decrease in GLU (Pre: 1.82±1.4 vs. Post: 1.09±0.3, p=0.10) while values reported for PLA increased (Pre: 1.09±0.3 vs. Post: 1.36±0.5, p=0.18). Additionally, within-group changes for ‘Hard Stools’ showed a larger decrease in GLU (Pre: 1.55±1.04 vs. Post: 1.09±0.3, p=0.10) when compared to changes observed in PLA (Pre: 1.27±0.65 vs. Post: 1.09±0.3, p=0.41). No other GSRS variables exhibited changes between groups as a result of supplementation. Notably, other GSRS variables for both PLA and GLU exhibited tendencies (p=0.05 to 0.10) to change from their baseline scores. ‘Constipation Syndrome’ exhibited a trend for the changes observed between GLU and PLA to be different (p=0.06). In this respect, changes in GLU tended to decrease from baseline (Pre: 5.09±3.45 vs. Post: 3.27±0.9, p=0.07) while no change was observed in PLA (Pre: 3.73±1.27 vs. Post: 3.64±0.81, p=0.93). Interestingly, both PLA and GLU observed similar increases (a negative change) in the ingestion syndrome (p=0.07 for both GLU and PLA), but no between-group changes were observed (p=0.88). All other GSRS changes are provided in Tables 2A and 2B. One male participant during the Glucosamine condition self-reported increased frequency and severity of diarrhea secondary to international travel during his supplementation protocol. His results have not been removed from the analysis.

TABLE 2A
Bristol Stool Chart and Gastrointestinal Symptom Rating Scale

| | PLA | | | GLU | | |
|---|---|---|---|---|---|---|
| | Pre | Post | p | Pre | Post | p |
| Bristol Stool | 3.1 ± 0.8 | 4.0 ± 1.4 | 0.10 | 3.3 ± 0.8 | 3.6 ± 0.8 | 0.10 |
| GSRS1 | 1.3 ± 0.6 | 1.5 ± 0.7 | 0.08 | 1.2 ± 0.4 | 1.5 ± 0.7 | 0.18 |
| GSRS2 | 1.0 ± 0.0 | 1.1 ± 0.3 | 0.32 | 1.1 ± 0.3 | 1.2 ± 0.4 | 0.56 |
| GSRS3 | 1.0 ± 0.0 | 1.0 ± 0.0 | 1.00 | 1.0 ± 0.0 | 1.2 ± 0.4 | 0.16 |
| GSRS4 | 1.55 ± 0.69 | 1.18 ± 0.4 | 0.05 | 1.82 ± 0.98 | 1.18 ± 0.4 | 0.08 |
| GSRS5 | 1.5 ± 1.21 | 1.3 ± 0.47 | 0.71 | 1.27 ± 0.9 | 1.55 ± 1.21 | 0.32 |
| GSRS6 | 1.1 ± 0.3 | 1.2 ± 0.4 | 0.32 | 1.5 ± 0.69 | 2.2 ± 1.3 | 0.10 |
| GSRS7 | 1.7 ± 1.27 | 2.4 ± 1.29 | 0.03 | 1.9 ± 1.22 | 2.1 ± 1.22 | 0.32 |
| GSRS8 | 1.0 ± 0.0 | 1.1 ± 0.3 | 0.32 | 1.2 ± 0.4 | 1.2 ± 0.4 | 1.0 |
| GSRS9 | 2.09 ± 1.14 | 2.64 ± 1.47 | 0.25 | 1.82 ± 0.75 | 2.46 ± 1.21 | 0.08 |
| GSRS10 | 1.09 ± 0.3 | 1.36 ± 0.5 | 0.18 | 1.82 ± 1.4 | 1.09 ± 0.3 | 0.10 |
| GSRS11 | 1.0 ± 0.0 | 1.18 ± 0.4 | 0.16 | 1.0 ± 0.0 | 1.73 ± 1.56 | 0.11 |
| GSRS12 | * * * | * * * | * * * | * * * | * * * | * * * |
| GSRS13 | 1.27 ± 0.65 | 1.09 ± 0.3 | 0.41 | 1.55 ± 1.04 | 1.09 ± 0.3 | 0.10 |
| GSRS14 | 1.0 ± 0.0 | 1.09 ± 0.3 | 0.32 | 1.27 ± 0.9 | 1.55 ± 0.93 | 0.08 |
| GSRS15 | 1.36 ± 0.67 | 1.18 ± 0.4 | 0.48 | 1.73 ± 1.1 | 1.09 ± 0.3 | 0.07 |
| Diarrhea | 2.0 ± 0.0 | 2.27 ± 0.65 | 0.18 | 2.27 ± 0.9 | 3.27 ± 1.56 | 0.02 |
| Ingestion | 5.91 ± 2.12 | 7.27 ± 2.83 | 0.09 | 6.45 ± 2.5 | 7.91 ± 2.98 | 0.07 |
| Constipation | 3.73 ± 1.27 | 3.64 ± 0.81 | 0.93 | 5.09 ± 3.45 | 3.27 ± 0.9 | 0.07 |
| Abdom Pain | 4.27 ± 1.85 | 4.0 ± 1.18 | 0.55 | 4.27 ± 1.68 | 4.18 ± 1.66 | 0.67 |
| Reflux | 2.0 ± 0.0 | 2.09 ± 0.3 | 0.32 | 2.09 ± 0.3 | 2.36 ± 0.67 | 0.18 |

TABLE 2B
Bristol Stool Chart and Gastrointestinal Symptom Rating Scale

PLA vs. GLU

| | p* | % | d | 95% CI |
|---|---|---|---|---|
| Bristol Stool | 0.20 | −19.9 | −0.61 | (−1.83, 0.74) |
| GSRS1 | 1.00 | 31.3 | 0.69 | (−0.42, 0.42) |
| GSRS2 | 1.00 | −0.9 | 0.00 | (−0.30, 0.30) |
| GSRS3 | 0.16 | 20.0 | 1.00 | (−0.09, 0.45) |
| GSRS4 | 0.45 | −11.3 | −0.41 | (−1.07, 0.53) |
| GSRS5 | 0.33 | 35.4 | 0.48 | (−0.46, 1.37) |
| GSRS6 | 0.16 | 37.6 | 0.77 | (−0.32, 1.41) |
| GSRS7 | 0.03 | −30.7 | −0.40 | (−0.81, −0.10) |
| GSRS8 | 0.56 | −10 | −0.31 | (−0.45, 0.27) |
| GSRS9 | 0.87 | 8.8 | 0.08 | (−1.51, 1.69) |
| GSRS10 | 0.10 | −64.9 | −1.29 | (−2.20, 0.20) |
| GSRS11 | 0.29 | 55.0 | 0.68 | (−0.55, 1.64) |
| GSRS12 | *** | *** | *** | *** |
| GSRS13 | 0.08 | −15.5 | −0.43 | (−0.59, 0.04) |
| GSRS14 | 0.16 | 13.0 | 0.29 | (−0.09, 0.45) |
| GSRS15 | 0.20 | −23.8 | −0.67 | (−1.21, 0.30) |
| Diarrhea | 0.13 | 30.6 | 0.76 | (−0.36, 1.81) |
| Ingestion | 0.88 | −0.4 | 0.04 | (−2.52, 2.70) |
| Constipation | 0.06 | −33.3 | −0.89 | (−3.70, 0.24) |
| Abdom Pain | 0.86 | 4.2 | 0.11 | (−1.05, 1.41) |
| Reflux | 0.16 | 8.4 | 0.45 | (−0.09, 0.45) |

Values inside tables 2A and 2B are means±standard deviations for each specified variable and timepoint. Bold-type face=Between-group difference (p<0.05). *=p-value computed using non-parametric statistical approaches (Wilcoxon-signed rank test). GSRS1=Pain and discomfort in upper abdomen or stomach; GSRS2=Heartburn; GSRS3=Acid reflux; GSRS4=Hunger pangs; GSRS5=Nausea; GSRS6=Stomach rumbling; GSRS7=Stomach bloating; GSRS8=Burping; GSRS9=Passing gas or flatulence; GSRS10=Constipation; GSRS11=Diarrhea; GSRS 12=Was not assessed due to technical error; GSRS13=Hard stools; GSRS14=Urgent need to have a bowel movement; GSRS15=Sensation of not completely emptying the bowels; Diarrhea=Combined score of GSRS11 and GSRS14; Ingestion=Combined score of GSRS6, GSRS7, GSRS8, and GSRS9; Constipation=Combined score of GSRS10, GSRS13, and GSRS14; Abdom Pain=Combined score of GSRS 1, GSRS4, and GSRS5; Reflux=Combined score of GSRS2 and GSRS3.

### Example 4

### Fecal Microbiota Analysis

A fecal microbiota analysis was further conducted. DNA was extracted from fecal samples by mechanical lysis with a FastPrep-24 (MP Biomedicals, Burlingame, Calif.) and later purified using the DNA Fast Stool Minikit (Qiagen Inc USA, Germantown, Md.) according to previously described methods. The V4 region of bacterial 16S rRNA genes (294 bp) was amplified in 35 cycles according to previously described methods with Takara ExTaq (Takara Bio USA, Mountain View, Calif.) and the F515 (5′-GTGCCAGCMGCCGCGGTAA-3′) and R806 (5′-GGATACHVGGGTWTCTAAT-3′) primer containing random eight base pair barcode sequences on the 5′ end of the forward primer. The Qubit Fluorometer (Thermo Fisher Scientific, San Francisco, Calif.) was used to quantify PCR product concentrations prior to combining the PCR products in equal quantities for purification with the Wizard SV Gel and PCR Cleanup kit (Promega, Madison, Wis.). The lonTorrent Fragment Plus Library Kit (Thermo Fisher) with AMPure XP Magnetic Beads (Beckman Coulter Life Sciences, Pasadena, Calif.) was then used for DNA sequencing library preparation. Library purity and concentration was measured on a 2100 Bioanalyzer System (Agilent Technologies, Santa Clara, Calif.). The library was diluted to 20 pM and then sequenced with an Ion Genestudio S5 System (Thermo Fisher).

Ion Torrent BAM files of raw DNA sequencing data were converted to the FASTQ format using BEDTools and then analyzed using Quantitative Insights into Microbiology (QIIME2) version 2019.10. Reads shorter than 200 bp in length were removed. The sequences were demultiplexed using the cutadapt demux-single command, denoised using the QIIME deblur denoise-16S command, and truncated to a length of 270 bp to trim low q-score base positions. Multiple sequence Alignment using Fast Fourier Transform (MAFFT) was used for read alignment, while gapped and unconserved alignments were filtered using the alignment mask plugin. A phylogenetic tree was generated using FastTree, and taxonomic classification was performed against the Greengenes 13.8 database. Sequences of mitochondria and chloroplast origin were filtered from downstream analysis, leaving a total of 1,391,259 total high-quality reads associated with taxonomic annotations. Samples were rarified to 7,874 reads for alpha and beta diversity analysis. Two samples were eliminated from the downstream analysis (GLC_3_L_Pre, GLC_7_L_Post), due to having low levels of annotated reads (GLC_7_L_Post: 8.3% reads annotated past kingdom level, GLC_3_L_Pre: 42.7% reads annotated past kingdom level). Beta diversity metrics of Bray-Curtis distances and Weighted and Unweighted Unifrac distances were plotted using principal coordinate analysis (PCoA). Alpha diversity metrics of Observed OTUs, Shannon Evenness, and Faith's Phylogenetic Diversity were calculated.

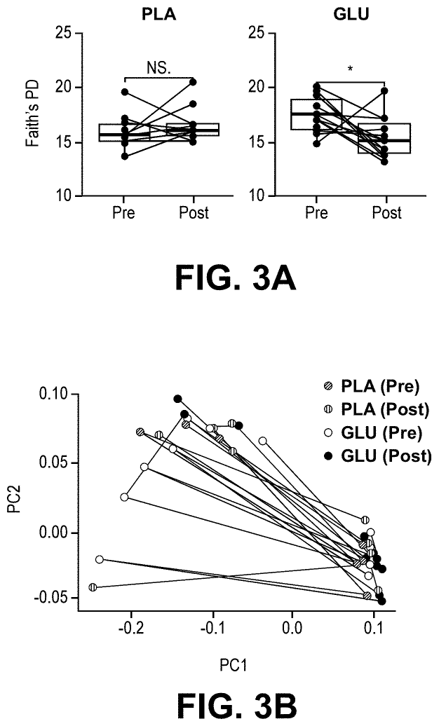

FIGS. 3A and 3B shows the intestinal microbiota diversity before and after glucosamine consumption. FIG. 3A shows the Faith's Phylogenetic Diversity (PD) of bacterial contents in fecal samples before and after PLA and GLU consumption, with points representing individual fecal sample communities. Bacterial composition before and after GLU treatment was significantly different (Student t-test, p<0.05). FIG. 3B provides the Principal Components Analysis (PCoA) of bacterial diversity according to the unweighted UniFrac metric. Fecal samples from each individual are connected with black lines. No significant difference was detected based on treatment subgroup (PERMANOVA, p>0.05). Further, longitudinal analysis showed that the alpha diversity of the bacterial communities in the fecal contents was significantly decreased following GLU consumption compared to baseline values (Faith's Phylogenetic Diversity (PD), paired t-test, treatment+time, p=4.59E-03). By comparison, Faith's PD did not change over time when PLA was consumed (See FIG. 3A). The other alpha diversity measures (Observed OTUs or Shannon Evenness) were not significantly altered by the inclusion of either GLU or PLA into the diet (p>0.05, paired t-test). Although bacterial beta diversity as assessed by unweighted Unifrac distances showed that the fecal microbiota changed over time between fecal samples collected before and after GLU or PLA consumption (See FIG. 3B), the changes in beta-diversity were not significantly different compared to baseline (PERMANOVA, p>0.05). There were also no significant differences in fecal microbiota diversity according to the weighted Unifrac and Bray-Curtis Dissimilarity metrics (p>0.05, PERMANOVA).

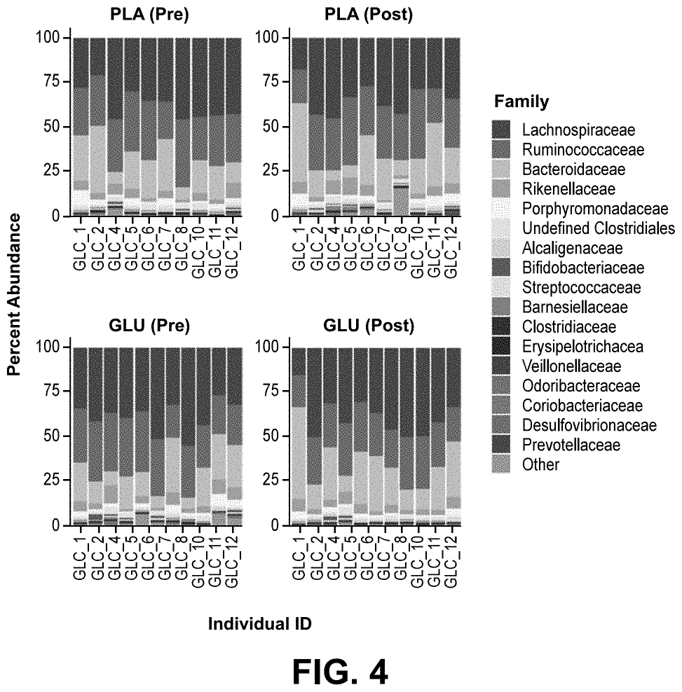

Lachnospiraceae and Ruminococcaceae (Firmicutes) and Bactroidaceae (Bacteroidetes) families comprised the majority of bacterial taxa present in the fecal contents (84.8±4.7% combined relative abundance). FIG. 4 shows the relative abundance of bacteria shown at the family level. Proportions of individual bacterial families pre and post GLU and PLA consumption are shown in FIG. 4. ASVs were combined if their family-level taxonomic designations were identical. The seventeen most abundant bacterial families are annotated, with all other taxa grouped under the annotation of “Other”. No other bacterial families were present at an average relative abundance higher than 5% in any of the fecal samples.

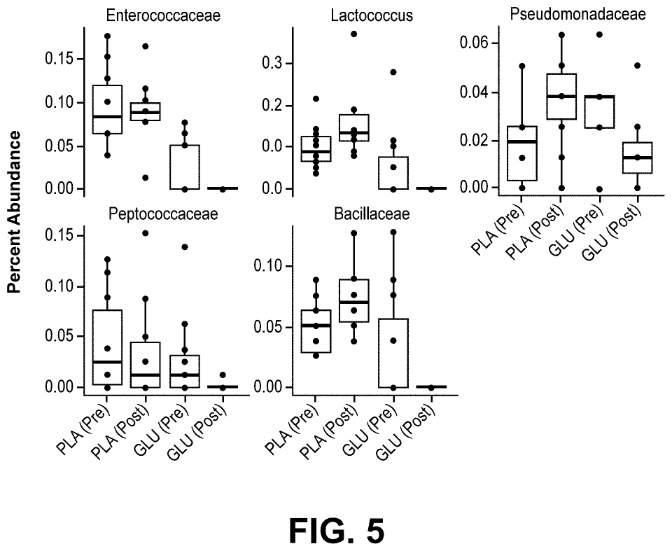

FIG. 5 shows the bacterial taxa present in significantly different proportions in fecal contents before and after PLA and GLU supplementation. For the graphs showing the percent abundance for Enterococcaceae and Lactococcus, the data indicates that these taxa were present in significantly different proportions between PLA and GLU (PRE). For the graph showing the percent abundance for Pseudomonadaceae, the data denotes a significant difference between GLU (Pre) and GLU (Post). For the graphs showing the percent abundance for Peptococcaceae and Bacillaceae, the data denotes a significant difference for GLU (Post) compared to GLU (Pre) as well as to both PLA (Pre) and PLA (Post). Taxa were compared using MaAsLin (p<0.05). Comparisons between the fecal microbiota contents showed that the levels of Enterococcaceae and Lactococcus were variable, but not in a manner that was associated with GLU consumption. Among the other taxa identified, lower proportions of Pseudomonadaceae (p=0.014, Log₂ FC=−1.05), Peptococcaceae (p=0.021, Log₂ FC=−4.52), and Bacillaceae (p=0.038, not detected post-GLU), and were found after GLU intake compared to baseline (GLU (Pre)). By comparison, there were no significant differences in bacterial composition after PLA ingestion compared to PLA (Pre) (MaAsLin p>0.05). Additionally, levels of Peptococcaceae and Bacillaceae were also significantly reduced after GLU intake compared to either before (PLA (Pre)) or after PLA (PLA (Post)) consumption. While the proportions of those two families were also lower at GLU (Pre) baseline compared to the PLA (Post) time point, those differences were not significant (MaAsLin, p>0.05).

As shown within the results, significant reductions in the proportions of Pseudomonadaceae, Peptococcaceae, and Bacillaceae in the fecal contents were found when glucosamine was consumed compared to baseline samples. It is also notable that the levels of Peptococcaceae and Bacillaceae were also significantly reduced after GLU compared to before and after PLA consumption, thereby indicating a longitudinal and treatment specific effect. Conversely, despite reductions in the proportions of those taxa, there was no single bacterial genus or family that was enriched. These results may be due to glucosamine-mediated inhibition of certain bacterial taxa and the concurrent (non-specific) enrichment of multiple bacterial species able to metabolize this compound. The cross-over, longitudinal design study and inclusion of baseline samples within the example demonstrated how the bacterial contents within a single individual can change over time. Comparisons of the baseline samples indicated significant variations in the abundance of Enterococcaceae and Lactococcus suggesting that other factors may determine the levels of those taxa in the distal intestine.

### Example 5

### Fecal Metabolome Analysis

A Fecal Metabolome Analysis was further conducted. Samples were manually homogenized with a sterile microspatula, and approximately 250 mg of fecal material was weighed and extracted using 1.5 mL of ice-cold Dulbecco's phosphate buffered saline (DPBS, 1×, pH 7.4). After extraction, samples were centrifuged, and the pellet was dried using a Labconco FreeZone 4.5 L Freeze Dry System to determine fecal dry weight. The supernatant was sequentially filtered through a 0.22 μm pore-size syringe filter (Millex-GP syringe filter, Millipore, Billerica, Mass.) to remove microbes followed by a 3 kDa ultracentrifugal filter (Amicon ultracentrifugal filter, Millipore, Billerica, Mass.) to remove any excess proteins. To 207 L of the filtrate, 23 L of an internal standard (5 mM DSS-d6 (trimethylsilylpropanesulfonate) containing 0.2% NaN3 in 99.8% D20 was added to aid in quantification of metabolites. The pH of each sample was adjusted to 6.8±0.1 through addition of small amounts of NaOH or HCl. Samples were transferred to 3 mm Bruker NMR tubes (Bruker, Brillerica, Mass.) and stored at 4° C. until spectral acquisition. Nuclear magnetic resonance (NMR) spectra were acquired using a Bruker Avance 600 MHz NMR spectrometer equipped with a SampleJet autosampler (Bruker BioSpin, Germany) at 298 K using the NOESY 1H presaturation experiment (‘noesypr1d’). Data were acquired with a spectral width of 12 ppm, a 2.5-second acquisition time, a relaxation delay of 2.5 seconds, and a 100-millisecond mixing time using 32 transients and 8 dummy scans. Water saturation was applied during the relaxation delay and mixing time. Each spectrum was Fourier Transformed with zero filling to 128,000 data points, and the resulting Free Induction Decay (FID) was transformed with an exponential apodization function corresponding to a line-broadening of 0.5 Hz. Spectra were subsequently phased and baseline corrected using Chenomx NMRSuite Processor v8.3 (Chenomx Inc., Edmonton, Canada). Metabolites were identified and quantified based on the known concentration of the internal standard, using Chenomx NMRSuite Profiler v8.3 based on the established method of targeted profiling.

As shown within Table 3, GLU and PLA supplementation had no effect on individual or total short-chain fatty acids (p>0.05).

TABLE 3
Fecal Short-Chain Fatty Acids

| | | | | | | PLA vs. GLU | | | |
|---|---|---|---|---|---|---|---|---|---|
| | | PLA | p* | GLU | p* | p^{‡} | % | d | 95% CI |
| Formate | Pre | 244 ± 116 | 0.56 | 298 ± 166 | 0.20 | 0.25 | 30.7 | 0.49 | (−270, 81) |
| | Post | 255 ± 135 | | 403 ± 299 | | | | | |
| Acetate | Pre | 55,604 ± 17,431 | 0.70 | 40,675 ± 12,876 | 0.46 | 0.90 | 0.5 | −0.05 | (−14,026, 15,644) |
| | Post | 59,417 ± 22,185 | | 43,680 ± 11,351 | | | | | |
| Propionate | Pre | 15,419 ± 8193 | 0.86 | 12,596 ± 4145 | 0.96 | 0.48 | −10.0 | −0.22 | (−3303, 6411) |
| | Post | 17,040 ± 9419 | | 12,663 ± 5766 | | | | | |
| Butyrate | Pre | 15,244 ± 9517 | 0.87 | 11,370 ± 5781 | 0.14 | 0.51 | 14.3 | 0.19 | (−6234, 3366) |
| | Post | 15,978 ± 7809 | | 13,538 ± 6558 | | | | | |
| Isobutyrate | Pre | 1787 ± 984 | 0.52 | 2097 ± 887 | 0.73 | 0.11 | −38.6 | −0.59 | (−193, 1612) |
| | Post | 2356 ± 1474 | | 1956 ± 1334 | | | | | |
| Isovalerate | Pre | 1542 ± 967 | 0.68 | 1885 ± 886 | 0.67 | 0.09 | −18.9 | −0.33 | (−365, 1377) |
| | Post | 1956 ± 1226 | | 1712 ± 1304 | | | | | |
| Valerate | Pre | 2674 ± 1362 | 0.80 | 2691 ± 1314 | 0.78 | 0.22 | −36.0 | −0.53 | (−110, 1283) |
| | Post | 3063 ± 1833 | | 2574 ± 1519 | | | | | |
| Total SCFA | Pre | 92,514 ± 35,299 | 0.80 | 71,612 ± 22,891 | 0.49 | 0.81 | −1.3 | −0.08 | (−21,884, 27,156) |
| | Post | 100,064 ± 41,479 | | 76,527 ± 25,492 | | | | | |

Values inside table are means ± standard deviations for each specified variable and time point.
*= p-value computed using paired samples t-test.
^{‡}p-value computed using 2 × 2 within-within factorial ANOVA.

As shown in Table 4, compared to baseline, GLU supplementation significantly reduced fecal glutamate, isoleucine, leucine and valine content (p<0.05), as well as total branched-chain amino acid (−24%, p<0.05), and total amino acids (−21%, p<0.05). No significant changes were observed for any of the individual or total amino acids in the control group (p>0.05).

TABLE 4
Fecal Amino Acids

| | | | | | | PLA vs. GLU | | | |
|---|---|---|---|---|---|---|---|---|---|
| | | PLA | p* | GLU | p* | p^{‡} | % | d | 95% CI |
| Alanine | Pre | 2503 ± 1098 | 0.62 | 2667 ± 1134 | 0.16 | 0.24 | −33.4 | −0.72 | (−688, 2415) |
| | Post | 2928 ± 1636 | | 2228 ± 721 | | | | | |
| Glutamate | Pre | 7857 ± 2856 | 0.68 | 8882 ± 3831 | 0.04 | 0.01 | −30.7 | −0.90 | (801, 4504) |
| | Post | 8400 ± 2641 | | 6773 ± 2223 | | | | | |
| Glycine | Pre | 1082 ± 438 | 0.63 | 1207 ± 514 | 0.09 | 0.22 | −34.5 | −0.76 | (−293, 1091) |
| | Post | 1238 ± 684 | | 965 ± 399 | | | | | |
| Isoleucine | Pre | 904 ± 402 | 0.50 | 1029 ± 462 | 0.05 | 0.17 | −40.5 | −0.84 | (−202, 995) |
| | Post | 1049 ± 603 | | 777 ± 388 | | | | | |
| Leucine | Pre | 1303 ± 611 | 0.68 | 1422 ± 622 | 0.05 | 0.23 | −28.4 | −0.64 | (−307, 1101) |
| | Post | 1372 ± 737 | | 1094 ± 496 | | | | | |
| Lysine | Pre | 1658 ± 748 | 0.59 | 1834 ± 815 | 0.11 | 0.07 | −32.1 | −0.73 | (−56, 1194) |
| | Post | 1847 ± 943 | | 1454 ± 554 | | | | | |
| Methionine | Pre | 374 ± 367 | 0.84 | 424 ± 290 | 0.15 | 0.55 | −23.7 | −0.18 | (−263, 458) |
| | Post | 399 ± 292 | | 352 ± 210 | | | | | |
| Phenylalanine | Pre | 625 ± 311 | 0.70 | 629 ± 280 | 0.26 | 0.52 | −15.6 | −0.34 | (−237, 434) |
| | Post | 638 ± 331 | | 544 ± 208 | | | | | |
| Proline | Pre | 709 ± 304 | 0.99 | 687 ± 280 | 0.63 | 0.85 | −5.7 | −0.12 | (−413, 493) |
| | Post | 716 ± 383 | | 654 ± 326 | | | | | |
| Threonine | Pre | 768 ± 311 | 0.53 | 831 ± 355 | 0.14 | 0.16 | −31.8 | −0.73 | (−127, 641) |
| | Post | 853 ± 455 | | 659 ± 252 | | | | | |
| Tyrosine | Pre | 789 ± 468 | 0.60 | 874 ± 394 | 0.14 | 0.16 | −30.5 | −0.66 | (−128, 640) |
| | Post | 879 ± 387 | | 707 ± 273 | | | | | |
| Valine | Pre | 1180 ± 507 | 0.46 | 1351 ± 610 | 0.03 | 0.15 | −42.4 | −0.90 | (−239, 1329) |
| | Post | 1376 ± 791 | | 1003 ± 451 | | | | | |
| Total BCAAs | Pre | 3386 ± 1502 | 0.54 | 3802 ± 1674 | 0.04 | 0.18 | −36.5 | −0.80 | (−3413, 735) |
| | Post | 3796 ± 2123 | | 2873 ± 1312 | | | | | |
| Total AA | Pre | 19,752 ± 7608 | 0.59 | 21,838 ± 8104 | 0.02 | 0.06 | −31.0 | −0.89 | (−246, 13,390) |
| | Post | 21,694 ± 8345 | | 17,208 ± 5190 | | | | | |

Values inside table are means ± standard deviations for each specified variable and time point.
Bold-type face = Between-group difference (p < 0.05).
*= p-value computed using paired samples t-test.
^{‡}p-value computed using 2 × 2 within-within factorial ANOVA.

As shown in Table 5, PLA supplementation did not result in any significant changes of miscellaneous fecal metabolites (p>0.05). GLU supplementation significantly reduced nucleotides compared to baseline (uracil, p=0.02), and between groups (ribose, p=0.01 and uracil, p=0.04).

No GLU was detected in either group in the fecal metabolome analysis. The origin of N-acetylglucosamine in the feces is not clear and is likely not derived from the supplementary glucosamine, as N-acetylglucosamine is made by bacteria and used for cell wall synthesis, or by the host, and is part of the extracellular matrix. Therefore, the origin of this compound in the feces is not clear. Lower BCAA, total amino acids, and glutamate could indicate increased absorption of those nutrients, increased utilization by microbes, or decreased production, assuming that some of the amino acids are coming directly from gut bacteria. Additionally, reduced nitrogen excretion could indicate reduced muscle protein breakdown, potentially due to reduced inflammation. As there is a well-established link between reduced nitrogen excretion (through fecal material) and reduced muscle protein breakdown, increased muscle breakdown results in excretion of amino acids in the feces, thus less amino acids (nitrogen) in the feces signals that more amino acids are retained in the body. Further, the data suggests that glucosamine results in loss of fat. Thus, administration of glucosamine results in improved body composition, including loss of fat while maintaining muscle mass. Gut microbiota-derived metabolites affect many biological processes of the host, including appetite control and weight management. As amino acids are among the gut microbiota-derived metabolites which have previously demonstrated alterations in obesity, the data indicates potential alternative health applications for glucosamine.

TABLE 5
Misc. Fecal Metabolites

| | | | | PLA vs. GLU | | | |
|---|---|---|---|---|---|---|---|
| PLA | p* | GLU | p* | p^{‡} | % | d | 95% CI |

Amino Acid Breakdown Products

| | | | | | | | | | |
|---|---|---|---|---|---|---|---|---|---|
| Cadaverine | Pre | 157 ± 76 | 0.30 | 170 ± 52 | 0.80 | 0.23 | −29.1 | −0.73 | (−35, 128) |
| | Post | 198 ± 66 | | 165 ± 56 | | | | | |
| Putrescine | Pre | 90 ± 84 | 0.69 | 92 ± 59 | 0.20 | 0.88 | 5.0 | 0.06 | (−79, 69) |
| | Post | 108 ± 120 | | 115 ± 61 | | | | | |
| p-Cresol | Pre | 464 ± 298 | 0.98 | 597 ± 330 | 0.72 | 0.29 | −27.7 | −0.34 | (−144, 421) |
| | Post | 557 ± 421 | | 551 ± 537 | | | | | |
| Urocanate | Pre | 103 ± 29 | 0.21 | 104 ± 57 | 0.30 | 0.17 | −38.7 | −0.81 | (−21, 100) |
| | Post | 124 ± 58 | | 85 ± 48 | | | | | |

Nucleotide Breakdown Products

| | | | | | | | | | |
|---|---|---|---|---|---|---|---|---|---|
| Hypoxanthine | Pre | 392 ± 224 | 0.73 | 414 ± 190 | 0.06 | 0.22 | −27.1 | −0.59 | (−83, 317) |
| | Post | 420 ± 222 | | 325 ± 143 | | | | | |
| Ribose | Pre | 1110 ± 552 | 0.07 | 1428 ± 760 | 0.65 | 0.01 | −37.4 | −0.77 | (158, 714) |
| | Post | 1450 ± 339 | | 1332 ± 533 | | | | | |
| Uracil | Pre | 866 ± 425 | 0.65 | 922 ± 352 | 0.02 | 0.04 | −34.7 | −0.81 | (26, 604) |
| | Post | 943 ± 469 | | 684 ± 287 | | | | | |

Microbially Produced Fermentation Products

| | | | | | | | | | |
|---|---|---|---|---|---|---|---|---|---|
| Ethanol | Pre | 1293 ± 2442 | 0.43 | 169 ± 98 | 0.04 | 0.33 | 123.7 | 0.71 | (−2940, 1108) |
| | Post | 481 ± 784 | | 272 ± 187 | | | | | |
| Fumarate | Pre | 175 ± 75 | 0.19 | 225 ± 151 | 0.84 | 0.40 | −29.4 | −0.43 | (−84, 190) |
| | Post | 221 ± 139 | | 218 ± 115 | | | | | |
| Lactate | Pre | 38 ± 20 | 0.47 | 60 ± 49 | 0.43 | 0.56 | 107.7 | 0.40 | (−394, 229) |
| | Post | 68 ± 82 | | 172 ± 396 | | | | | |
| Methionine- | Pre | 241 ± 166 | 0.42 | 258 ± 191 | 0.39 | 0.29 | 37.4 | −0.51 | (−95, 282) |
| sulfoxide | Post | 278 ± 222 | | 202 ± 149 | | | | | |
| Phenylacetate | Pre | 888 ± 577 | 0.78 | 1095 ± 511 | 0.70 | 0.22 | −30.6 | −0.4 | (−210, 791) |
| | Post | 1082 ± 772 | | 999 ± 786 | | | | | |
| Succinate | Pre | 924 ± 642 | 0.99 | 635 ± 289 | 0.48 | 0.94 | 8.0 | 0.03 | (−497, 462) |
| | Post | 1029 ± 712 | | 758 ± 430 | | | | | |

Likely Host Derived

| | | | | | | | | | |
|---|---|---|---|---|---|---|---|---|---|
| 3-Hydroxybutyrate | Pre | 74 ± 37 | 0.53 | 89 ± 62 | 0.41 | 0.98 | 4.3 | 0.02 | (−60, 58) |
| | Post | 60 ± 42 | | 76 ± 35 | | | | | |
| N-Acetylglucosamine | Pre | 260 ± 216 | 0.81 | 249 ± 147 | 0.16 | 0.56 | −34.9 | −0.48 | (−248, 425) |
| | Post | 286 ± 207 | | 187 ± 152 | | | | | |

Likely Diet Derived

| | | | | | | | | | |
|---|---|---|---|---|---|---|---|---|---|
| Glucosamine | Pre | n.d. | | n.d. | | | | | |
| | Post | n.d. | | n.d. | | | | | |
| Glucose | Pre | 3650 ± 3069 | 0.90 | 2229 ± 2054 | 0.23 | 0.38 | 61.4 | 0.59 | (−5329, 2252) |
| | Post | 3217 ± 2414 | | 3334 ± 2753 | | | | | |
| Glycerol | Pre | 6013 ± 2267 | 0.62 | 7458 ± 7347 | 0.47 | 0.55 | −33.0 | −0.40 | (−5965, 10,342) |
| | Post | 7141 ± 7055 | | 6397 ± 3575 | | | | | |
| Nicotinate | Pre | 371 ± 153 | 0.27 | 327 ± 162 | 0.23 | 0.76 | −6.6 | −0.13 | (−105, 139) |
| | Post | 332 ± 95 | | 271 ± 106 | | | | | |
| Xylose | Pre | 594 ± 317 | 0.83 | 441 ± 420 | 0.31 | 0.23 | 70.8 | 0.82 | (−912, 250) |
| | Post | 525 ± 271 | | 702 ± 545 | | | | | |

Values inside table are means ± standard deviations for each specified variable and time point.
Bold-type face = Between-group difference (p < 0.05).
*= p-value computed using paired sample t-test.
^{‡}p-value computed using 2 × 2 within-within factorial ANOVA.
n.d. = non detectable.

While the present disclosure has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the disclosure.

What is claimed is:

1. A method of increasing nitrogen retention in a subject comprising:
administering to the subject a therapeutically effective amount of a composition comprising a glucosamine,
wherein the administering of the composition increases nitrogen retention in the subject.

2. The method of claim 1, wherein the subject is administered a total daily dose of between about 500 mg and about 5,000 mg of the glucosamine.

3. The method of claim 2, wherein the composition is administered one or more times per day.

4. The method of claim 2, wherein the composition is administered up to three times per day.

5. The method of claim 2, wherein the composition is administered to the subject for a period of at least 21 days.

6. The method of claim 1, wherein the administering of the composition reduces amino acid excretion via stools of the subject.

7. The method of claim 1, wherein the administering of the composition reduces a rating of stomach bloating by the subject according to Gastrointestinal Symptom Rating Scale 7 (GSRS).

8. The method of claim 1, wherein the glucosamine is not derived from shellfish.

9. The method of claim 1, wherein the composition is not co-administered with chondroitin or green lipped muscle extract to the subject.

10. A method of preventing, eliminating, and/or reducing bacterial diversity within a gastrointestinal microbiome of a subject comprising:
administering to the subject a therapeutically effective amount of a composition comprising a glucosamine,
wherein the administering of the composition prevents, eliminates, and/or reduces bacterial diversity within the gastrointestinal microbiome of the subject.

11. The method of claim 10, wherein the subject is administered a total daily dose of between about 500 mg and about 5,000 mg of the glucosamine.

12. The method of claim 11, wherein the composition is administered one or more times per day.

13. The method of claim 11, wherein the composition is administered up to three times per day.

14. The method of claim 11, wherein the composition is administered to the subject for a period of at least 21 days.

15. The method of claim 10, wherein the administering of the composition reduces the percent abundance of Pseudomonadaceae, Peptococcaceae, Bacillaceae, or a combination thereof within the gastrointestinal microbiome of the subject.

16. The method of claim 10, wherein the administering of the composition reduces a rating of stomach bloating by the subject according to Gastrointestinal Symptom Rating Scale 7 (GSRS).

17. The method of claim 10, wherein the glucosamine is not derived from shellfish.

18. The method of claim 10, wherein the composition is not co-administered with chondroitin or green lipped muscle extract to the subject.

19. An oral composition for improving gastrointestinal function in a subject comprising:
a source of glucosamine selected from the group consisting of glucosamine hydrochloride, glucosamine sulfate, and N-acetylglucosamine; and
a pharmaceutically acceptable carrier,
wherein the source of glucosamine is not derived from shellfish.

20. The composition of claim 19, wherein the composition is substantially free of chondroitin and green lipped muscle extract.
